SEQUENCE LISTING (1 sequence)

SEQ ID NO: 1 - protein, 4 aa
AAPF

(19) United States
(12) Patent Application Publication
(10) Pub. No.: US 2004/0214157 A1
(43) Pub. Date: Oct. 28, 2004

(54) CHROMATOGRAPHIC RESINS AND METHODS FOR USING SAME

(76) Inventors: SIMON C. BURTON, PALMERSTON NORT (NZ); DAVID R.K. HARDING, PALMERSTON NORT (NZ); NATHANIEL T. BECKER, BURLINGAME, CA (US); BEN A. BULTHUIS, SAN BRUNO, CA (US); LANDON M. STEELE, SAN FRANCISCO, CA (US)

Correspondence Address: BURNS DOANE SWECKER & MATHIS L L P, POST OFFICE BOX 1404, ALEXANDRIA, VA, 22313-1404, US

(21) Appl. No.: 08/468,610

(22) Filed: Jun. 6, 1995

Related U.S. Application Data
(62) Division of application No. 08/268,178, filed on Jun. 29, 1994, now abandoned.

(51) Int. Cl. C12Q 1/70; C12N 9/48

(52) U.S. Cl. 435/5

(57) ABSTRACT

Disclosed are resins, resin-protein/peptide complexes and methods for purifying proteins and peptides using said resins. The resins described herein are useful for the binding of a selected protein or peptide, particularly from an aqueous medium such as a fermentation broth, by hydrophobic interactions between the resin and the selected protein or peptide. The resin is characterized by the fact that it contains ionizable ligands and/or functionalities which are uncharged at the pH of binding the target protein or peptide, thereby facilitating hydrophobic interactions, and charged at the pH of desorption, thereby disrupting the established hydrophobic interaction between the resin and the target protein or peptide. More particularly, the present invention is directed to the use of the described resins in the purification of recombinant enzyme products such as proteases, for example, chymosin or subtilisin.

### BACKGROUND OF THE INVENTION

1. Field of the Invention

This invention relates to complexes of chromatographic resins with proteins and peptides and methods for purifying proteins or peptides using such resins. In particular, the chromatographic resins described herein are useful for the binding of a target protein or peptide, particularly from an aqueous medium such as a fermentation broth, by hydrophobic interactions between the resin and the target protein or peptide. The resin is characterized by the fact that it contains ionizable functionalities which are electrostatically uncharged at the pH of binding the target protein or peptide, thereby facilitating hydrophobic interactions, and charged at the pH of desorption, thereby disrupting the established hydrophobic interaction between the resin and the target protein or peptide.

### REFERENCES

The following references are referred to in the specification as numbers in [ ]:

1 Ochoa, J. L., “Hydrophobic (interaction) chromatography”, Biochimie, 60:1-15 (1978).

2 Yon, R. J., et al., “Protein Chromatography on Adsorbents with Hydrophobic and Ionic Groups”. Biochem J., 151:281-290 (1975).

3 Yon R. J., “Chromatography of Lipophilic Proteins on Adsorbents Containing Mixed Hydrophobic and Ionic Groups”, Biochem J., 126:765-767 (1972).

4 Hoftsee, B. H. J., “Hydrophobic Affinity Chromatography of Proteins”, Anal. Biochem. 52:430-448 (1973).

5 Hoftsee, B. H. J., “Protein Binding by Agarose Carrying Hydrophobic Groups in Conjunction with Charges”, Biochem. Biophys. Res. Commun., 50:751-757 (1973).

6 Jost, R. et al., “The Mode of Adsorption of Proteins to Aliphatic and Aromatic Amines Coupled to Cyanogen Bromide-Activate Agarose”, Biochem. Biophys., 362:75-82 (1974).

7 Kasche V., et al., “Rapid Protein Purification Using Phenylbutylamine-Eupergit: a novel method for large-scale procedures”, J Chromatogr., 510: 149-154 (1990).

8 Sasaki I. et al., “Hydrophobic-Ionic Chromatography”, J. Biochem., 86:1537-1548 (1979).

9 Sasaki I. et al., “Hydrophobic-Ionic Chromatography: Its Application to Microbial and Glucose Oxidase, Hyaluronidase, Cholesterol Oxidase, and Cholesterol Esterase”, J. Biochem., 91:1555-1561 (1982).

10 Simons, P. C. et al., “Purification of Glutathione S-Transferases from Human Liver by Glutathione-Affinity Chromatography”, Anal. Biochem., 82:334-341 (1977).

11 Asenjo, J. A. et al., “Rational Design of Purification Processes for Recombinant Proteins”, Ann. N.Y. Acad. Sci., 646:334-356 (1991).

12 Butler. L. G., “Enzyme Immobilization by Adsorption on Hydrophobic Derivatives of Cellulose and Other Hydrophilic Materials”, Arch. Biochem. Biophys., 171:645-650 (1975).

13 Caldwell, K. D. et al., “Utilization of Hydrophobic Interaction for the Formation of an Enzyme Reactor Bed”, Biotechnol. Bioeng., 17:613-616 (1975).

14 Cashion, P. et al., “Enzyme Immobilization on Tritylagarose”, Biotech. Bioeng., 24:403-423 (1982).

15 Voutsinas, P. L. et al., “Coagulation of Skim Milk with Proteases Immobilized on Hydrophobic Carriers”, Dairy Sci., 66:694-703 (1983).

16 Hutchinson, D. W., “The Preparation and Properties of Immobilized Dipeptidyl-aminopeptidase I (cathepsin C)”, Biochim. Biophys. Acta, 916:1-4 (1987).

17 Ruaan, R. C. et al., “Dual-Functional Affinity Protein Purification”, Biotechnol. Prog., 4:107-112 (1988).

18 Teichberg, V. I., “Affinity-Repulsion Chromatography”, J. Chromatogr., 510:49-57 (1990).

19 Johansson, G. et al., “Affinity Partition Between Aqueous Phases —A Tool for Large-Scale Purification of Enzymes”, J. Biotechnol., 11:135-142 (1989).

20 Ortin, A. et al., “Large Scale Extraction of α-Lactalbumin and β-Lactoglobulin from Bovine Whey by Precipitation with Polyethylene Glycol and Partitioning in Aqueous Two-Phase Systems”, Prep. Biochem., 22:53-66 (1992).

21 Heath, C. A. et al., “Synthetic Membranes in Biotechnology: Realities and Possibilities”, Adv. Biochem. Eng./Biotechnol., 47:45-88 (1992).

22 Luong, J. H. T. et al., “Synthesis and Characterization of a Water-Soluble Affinity Polymer for Trypsin Purification”, Biotechnol. Bioeng., 31:439-446 (1988).

23 Champluvier, B. et al., “Dye-Ligand Membranes as Selective Adsorbents for Rapid Purification of Enzymes: A Case Study”, Biotechnol. Bioeng., 40: 33-40 (1992).

The disclosure of each of the above-referenced publications is hereby incorporated by reference in their entirety to the same extent as if each and every reference were individually incorporated by reference herein.

2. State of the Art

In recent years, several techniques have been developed and/or optimized to effect separation and purification of a selected protein or peptide from an aqueous mixture. The development of such techniques corresponds, in part, to the growth in recombinantly developed microorganisms which have been genetically modified to express a desired protein or peptide in a fermentation broth. Such broths, however, are characterized as containing not only the desired or target protein or peptide but a variety of other proteins or peptides expressed by that microorganism as well as other contaminants including, by way of example, whole cells where the target protein or peptide is extracellularly expressed and cell debris in those cases where the target protein or peptide is intracellularly expressed and lysis of the cell is required to extract the target protein or peptide into the aqueous medium.

Separation and purification techniques heretofore employed with proteins/peptides include, by way of example, ion-exchange chromatography, hydrophobic interaction chromatography, affinity chromatography, and the like. The multiplicity of such chromatographic techniques reflect the difficulty in effecting separation and/or purification without denaturing the selected protein or peptide while minimizing the complexity of the separation/purification procedure and each of the techniques recited above suffer from one or more drawbacks limiting their broad use on an industrial scale.

For example, in ion-exchange chromatography, good binding between the resin and the protein or peptide requires that the protein or peptide solution first be desalted typically by dilution, dialysis, diafiltration or gel filtration. Additionally, to effect removal of the bound protein or peptide from the resin, an aqueous solution containing a high salt concentration is typically contacted with the resin.

In hydrophobic interaction chromatography (HIC), the resin is ideally uncharged and binding is due solely to hydrophobic interactions. To effect protein or peptide binding to such resins, the protein or peptide solution is typically of high ionic strength [1]. Large quantities of salt such as ammonium sulfate or sodium chloride at molarities of greater than 1 molar are typically added to the protein or peptide solution to achieve the requisite high ionic strength. After binding, the bound protein or peptide is typically recovered by desalting.

Chromatographic techniques involving salting/desalting of the protein or peptide solution require the use of large quantities of reagents to effect recovery on an industrial level and may necessitate substantial processing. Accordingly, hydrophobic interaction chromatography and ion-exchange chromatography are not the most efficient and cost effective methods for recovering and/or purifying industrial quantities of a protein or peptide from a fermentation broth or other aqueous media.

Similarly, the use of affinity chromatography to effect separation and/or purification of a selected protein or peptide is limited in scope due to the rigorous process conditions required, the low throughput and high cost of this technique. Accordingly, this procedure is typically not amenable to the efficient recovery of industrial quantities of a protein or peptide from a fermentation broth or other aqueous media.

This invention is directed to the use of specific chromatographic resins which permits the surprising and efficient large scale recovery and/or purification of proteins or peptides from aqueous media including fermentation broths. The resins employed herein are characterized as comprising a solid support matrix and ligands covalently attached thereto wherein the resin is electrostatically uncharged at the pH where the target protein or peptide is bound to the resin and is electrostatically charged at the pH where the target protein or peptide is eluted from the resin. The resins described herein are further characterized as being capable of binding the target protein or peptide from a solution maintained at either high or low ionic strength.

In a preferred embodiment, the induced electrostatic charge on the resin at the pH where the target protein or peptide is desorbed from the resin is of the same polarity as the net electrostatic charge on the target protein or peptide at the desorption pH. In this embodiment, desorption is achieved by charge-charge repulsions which offset the hydrophobic binding character of the resin. In another embodiment, the induced electrostatic charge is of the opposite polarity from that of the target protein. In either embodiment, desorption can be facilitated by use of eluants of high ionic strength or by use of a polarity reducing agent, such as propylene glycol.

The resin-protein/peptide complexes of this invention are contrasted with resin-protein/peptide complexes heretofore described by the characteristic that the resin is electrostatically uncharged at the pH of protein binding and charged at the pH of desorption. Specifically, in the case of HIC resins, charged groups have been deliberately introduced [2,3] to weaken strong binding to long alkyl chain (hydrophobic) Sepharose resins and allow more favorable desorption conditions. These matrices contain positively charged isourea linkages and negatively charged carboxyl groups and will contain charged functionality at all pHs.

A polystyrene carboxyl resin (Amberlite) has also been used for protein binding by hydrophobic interactions [8,9]. Although said to be in an uncharged form at pHs less than 4.5 [8,9], titration experiments have shown that this matrix is only uncharged at pHs less than 3. In this system, proteins were bound at a pH of about 4.5 and eluted by a pH increase, which further deprotonates the charged matrix carboxyl groups and weakens hydrophobicity. In some cases, this causes electrostatic repulsion as different protein isoelectric points (IEPs) are passed. This method is useful only in a narrow pH range [9].

Also disclosed are resins which are positively charged with isourea groups [4,5]. These matrices are weakly hydrophobic and typically require electrostatic and hydrophobic interactions for binding of a protein or peptide to the resin. Moreover, the charged group in these resins is close to the solid support matrix not at the resin surface and a change of pH is not used for desorption of the protein or peptide from the resin. The desorption of proteins or peptides from the column has been shown to be easier when charged rather than uncharged resins are used [6]. Charged groups were not found to limit adsorption capacity while facilitating desorption from phenylbutylamine resins [7]. Adsorption was ascribed to hydrophobic interactions. The use of charged functionality to bind and then desorbed the proteins or peptides from the resin typically entails, however, adjustments in the ionic strength of the solution either prior to binding or to effect desorption. Such adjustments are not consistent with an efficient process to effect protein or peptide recovery and/or purification.

### SUMMARY OF THE INVENTION

This invention relates to complexes of resins with proteins and peptides and methods for purifying target proteins or peptides using such resins. The resins described herein have ionizable functionality and a solid support matrix wherein the resin is electrostatically uncharged at the pH of target protein or peptide binding to the resin and electrostatically charged at the pH of desorption. Because of the lack of charge on the resin at the binding pH, difficulties and/or complexities associated with, for example, ion exchange resins can be avoided.

In view of the above, in one of its composition aspects, this invention is directed to a resin-protein/peptide complex which comprises a resin and a target protein or peptide bound thereto wherein said resin comprises

a) a solid support matrix; and

b) selected ionizable ligand covalently attached to the matrix wherein the ionizable ligand is selected such that the resin is electrostatically uncharged at the pH where the target protein or peptide is bound to the resin and is electrostatically charged at the pH where the target protein or peptide is desorbed from the resin and further wherein about 40 percent or more and preferably 50 percent or more of the target protein or peptide in an aqueous medium binds to the resin when the aqueous medium has either a high or a low ionic strength.

In another of its composition aspects, this invention is directed to a resin-protein/peptide complex which comprises a resin and a target protein or peptide bound thereto wherein said resin comprises

a) a solid support matrix having a selected ionizable functionality incorporated into the backbone thereof wherein the ionizable functionality is selected such that the resin is electrostatically uncharged at the pH where the target protein or peptide is bound to the resin and is electrostatically charged at the pH where the target protein or peptide is desorbed from the resin; and

b) a non-ionizable ligand covalently attached thereto, wherein about 40 percent or more and preferably 50 percent or more of the target protein or peptide in an aqueous medium binds to the resin when the aqueous medium has either a high or a low ionic strength.

In one embodiment, the electrostatic charge induced on the resin in the resin-protein/peptide complex is of the same polarity as the net electrostatic charge on the target protein or peptide at the pH of desorption. In this embodiment, desorption is facilitated by charge-charge repulsions between the resin and the target protein or peptide.

In another embodiment, the electrostatic charge induced on the resin in the resin-protein/peptide complex is of the opposite polarity from the net electrostatic charge on the target protein or peptide at the pH of desorption. In this embodiment, desorption may be facilitated by, for example, the use of a desorbing solution of high ionic strength or the use of a polarity reducing agent, such as propylene glycol.

In one of its method aspects, this invention is directed to a method for separating a target protein or peptide from an aqueous medium comprising the target protein or peptide which method comprises contacting the medium with a resin as described above at a pH wherein the resin is electrostatically uncharged and under conditions sufficient to allow the target protein or peptide to bind to the resin wherein about 40 percent or more and preferably 50 percent or more of the target protein or peptide in the aqueous medium binds to the resin when the aqueous medium has either a high or a low ionic strength.

In another of its method aspects, this invention is directed to a method for binding and recovering a target protein or peptide from an aqueous medium comprising the target protein or peptide which method comprises:

a) contacting the medium with a resin as described above at a pH wherein the resin is electrostatically uncharged and under conditions sufficient to allow the target protein or peptide to bind to the resin wherein about 40 percent or more and preferably 50 percent or more of the target protein or peptide in the aqueous medium binds to the resin when the aqueous medium has either a high or a low ionic strength;

b) separating the resin containing the bound target protein or peptide from the other components of the medium to produce a resin-protein/peptide complex; and

c) desorbing the bound target protein or peptide from the complex by contacting the complex with a desorbing solution having a pH which induces an electrostatic charge on the resin wherein the induced charge is of the same polarity as the net charge on the target protein or peptide at the pH of the desorbing solution.

In still another of its method aspects, this invention is directed to a method for binding and recovering a target protein or peptide from an aqueous medium comprising the target protein or peptide which method comprises:

a) contacting the medium with a resin as described above at a pH wherein the resin is electrostatically uncharged and under conditions sufficient to allow the target protein or peptide to bind to the resin wherein about 40 percent or more and preferably 50 percent or more of the target protein or peptide in an aqueous medium binds to the resin when the aqueous medium has either a high or a low ionic strength;

b) separating the resin containing the bound target protein or peptide from the other components of the medium to produce a resin-protein/peptide complex; and

c) desorbing the bound target protein or peptide from the complex by contacting the complex with a desorbing solution having a pH which induces an electrostatic charge on the resin wherein the induced charge is of the opposite polarity from the net charge on the target protein or peptide at the pH of the desorbing solution. In this embodiment, desorption can be facilitated by, for example, use of a desorbing solution of high ionic, strength.

In either method embodiment, the use of a polarity reducing agent, such as propylene glycol, can facilitate desorption.

### BRIEF DESCRIPTION OF THE DRAWINGS

FIG. 1 illustrates ligand attachment to carbonyl diimidazole (CDI) activated matrices.

FIG. 2 illustrates carbodiimide coupling of ligand to matrix carboxyl groups.

FIG. 3 illustrates CDI-caproic acid supports.

FIG. 4 illustrates possible products from condensation (reaction) of an amine with a CDI-caproic acid matrix.

FIG. 5 illustrates reaction of an thiol with epoxy activated matrices.

FIG. 6 illustrates reaction of an amine with an epoxide activated matrix.

FIG. 7 illustrates the structure of some resins useful in the present invention.

FIGS. 8A-8K illustrate several configurations for the resins described herein.

### DETAILED DESCRIPTION OF THE INVENTION

This invention is directed, in part, to rational methods for recovering a target protein or peptide (hereafter sometimes collectively referred to as “protein”) from an aqueous medium using resins. The methods of this invention involve the selection of a resin for use in the chromatographic recovery of the target protein or peptide whieh resin is electrostatically uncharged at the pH where the target protein or peptide is bound to the resin and is electrostatically charged at the pH where the target protein or peptide is desorbed from the resin and further wherein about 50 percent or more of the target protein or peptide in an aqueous medium binds to the resin when the aqueous medium has either a high or a low ionic strength.

In one embodiment, an ionizable ligand is covalently attached to the matrix of the resin wherein the ionizable functionality of the ionizable ligand is selected so as to be electrostatically uncharged at the pH of target protein binding to the resin and charged at the pH of target protein desorption from the resin. This implies, of course, that the selection process is made relative to the properties of the target protein such as its p1, stability, etc. so that the pHs where the ionizable functionality is uncharged/charged corresponds to a pH range where the target protein is sufficiently stable, etc. One skilled in the art can readily ascertain such parameters based on the teachings herein.

In a preferred aspect of this embodiment, the selection process is extended to include the selection or the solid support matrix employed in conjunction with the ionizable ligand and the density of such ligands on the matrix wherein the selection is made relative to the degree of hydrophobicity required for the overall resin at the pHs of binding and desorption of the target protein from the resin. In this regard, the solid support matrix can be either free of ionizable functionality or can contain ionizable functionality which are electrostatically uncharged at the pH of target protein binding and electrostatically charged at the pH of target protein desorption. Additionally, the ionizable ligand can optionally comprise a spacer arm which, if employed, is selected to provide further control over the hydrophobicity at the pHs of protein binding and desorption.

In another embodiment, a solid support matrix having one or more selected ionizable functionalities incorporated into the backbone thereof and non-ionizable ligands attached thereto is employed for recovering the target protein from the aqueous medium. In this embodiment, the ionizable functionality is selected so as to be electrostatically uncharged at the pH of target protein binding to the resin and electrostatically charged at the pH of target protein desorption from the resin. This implies, of course, that the selection is made relative to the properties of the target protein such as its pI, stability, etc. so that the pHs where the ionizable functionality are uncharged/charged corresponds to a pH range where the target protein is sufficiently stable, etc. One skilled in the art can readily ascertain such parameters in view of the teachings herein.

In a preferred aspect of this embodiment, the selection process is extended to include the selection of one or more non-ionizable ligands relative to the degree of hydrophobicity required for the overall resin at the pHs of binding and desorption. For example, a more hydrophobic non-ionizable ligand can often be used to increase binding, if binding needs to be increased, whereas a less hydrophobic non-ionizable ligand often can be used to increase desorption or decrease non-specific binding if such is required. Additionally, the non-ionizable ligand can optionally comprise a spacer arm to link the non-ionizable ligand to the matrix which, when employed, is selected to provide further control over the hydrophobicity at the pH's of adsorption and desorption.

In either case, by making the appropriate selection for each of the components of the resin, the degree of resin hydrophobicity at the pH of target protein binding can be rationally selected to enhance the binding efficiency and/or increase binding specificity of the target protein or peptide to the resin. Likewise, the degree of hydrophilicity at the pH of target protein desorption from the resin, including at arising from the induced charge, can be rationally selected so as to ensure proper desorption of the target protein from the resin.

Prior to describing this invention in more detail, the following terms will first be defined.

Definitions

The term “solid support matrix” or “solid matrix” refers to the solid backbone material of the resin which material contains reactive functionality permitting the covalent attachment of ligand thereto. The backbone material can be inorganic (e.g., silica) or organic. When the backbone material is organic, it is preferably a solid polymer and suitable organic polymers are well known in the art. Solid support matrices suitable for use in the resins described herein include, by way of example, cellulose, regenerated cellulose, agarose, silica, coated silica, dextran, polymers (such as polyacrylates, polystyrene, polyacrylamide, polymethacrylamide including commercially available polymers such as Fractogel, Enzacryl, and Azlactone), copolymers (such as copolymers of styrene and divinylbenzene), mixtures thereof and the like. Also, co-, ter- and higher polymers can be used provided that at least one of the monomers contains or can be derivatized to contain a reactive functionality in the resulting polymer.

Reactive functionalities of the solid support matrix permitting covalent attachment of the ligand are well known in the art. Such groups include hydroxyl (e.g., Si—OH), carboxyl, thiol, amino, and the like. Conventional chemistry permits use of these functional groups to covalently attach ligands thereto. Additionally, conventional chemistry permits the inclusion of such groups on the solid support matrix. For example, carboxyl groups can be incorporated directly by employing acrylic acid or an ester thereof in the polymerization process. Upon polymerization, carboxyl groups are present if acrylic acid is employed or the polymer can be derivatized to contain carboxyl groups if an acrylate ester is employed.

The term “ionizable ligand” refers to a group covalently attached to the solid support matrix either directly or through a spacer arm which group contains one or more functionalities capable of being electrostatically charged at one pH and electrostatically uncharged at another pH. Suitable ionizable ligands include by way of example, amine groups, phenolic groups, carboxyl groups, histidyl groups, pyridyl groups, anilino groups, morpholinyl groups, imidazolyl groups, and the like. Substituents can be included on substitutable ligands for the purpose of modifying the pH at which these ligands will be electrostatically charged or uncharged. For example, inclusion of one or more nitro groups, halo groups, alkyl groups, etc. on a phenol group will change the pH at which this group will be electrostatically charged. Such modification of ligands is well within the skill of the art.

The term “selected ionizable ligand” refers to the ligand or mixture of ligands selected for covalent attachment to the solid support matrix. Selection of the ionizable ligand is made relative to the pH where the ligand will carry an induced electrostatic charge. In turn, the pHs selected for binding and desorption will depend on factors such as the pl and stability of the target protein. Accordingly, the rationale for selection of the appropriate ionizable ligand will be based, at least in part, on the use of an ionizable ligand that is electrostatically charged/uncharged under conditions that are compatible with the target protein to be recovered.

The term “a non-ionizable ligand” refers to a group covalently attached to the solid support matrix either directly or indirectly through a spacer arm which group does not contain an readily ionizable functionality. Suitable non-ionizable ligands include, by way of example, alkyl groups, aromatic groups (e.g., phenyl, naphthyl) and alkylaromatic groups (e.g. benzyl groups).

As noted above, the ionizable and non-ionizable ligands are either directly and covalently linked to the solid support matrix or these ligands comprise a spacer arm for covalently linking the ionizable functionality to the solid support matrix. Accordingly, attachment of an ionizable ligand to the solid support matrix can be illustrated as follows:

Solid Support Matrix-Ionizable Ligand

Insofar as the ligand can comprise a spacer arm, the above formula can be further illustrated as:

Solid Support Matrix-[Spacer Arm]ₙ-R

where n is 0 or 1; R is an ionizable functionality and Spacer Arm is a chemical group capable of covalently linking the ionizable functionality to the solid support matrix. The spacer arm can be devoid of ionizable functionalities or can contain one or more ionizable functionalities, e.g., histidyl, imidazolyl, etc.

Non-ionizable ligands are similar to the ionizable ligands described above except that R is replaced by R¹ which does not contain an ionizable functionality and Spacer Arm is devoid of ionizable functionality.

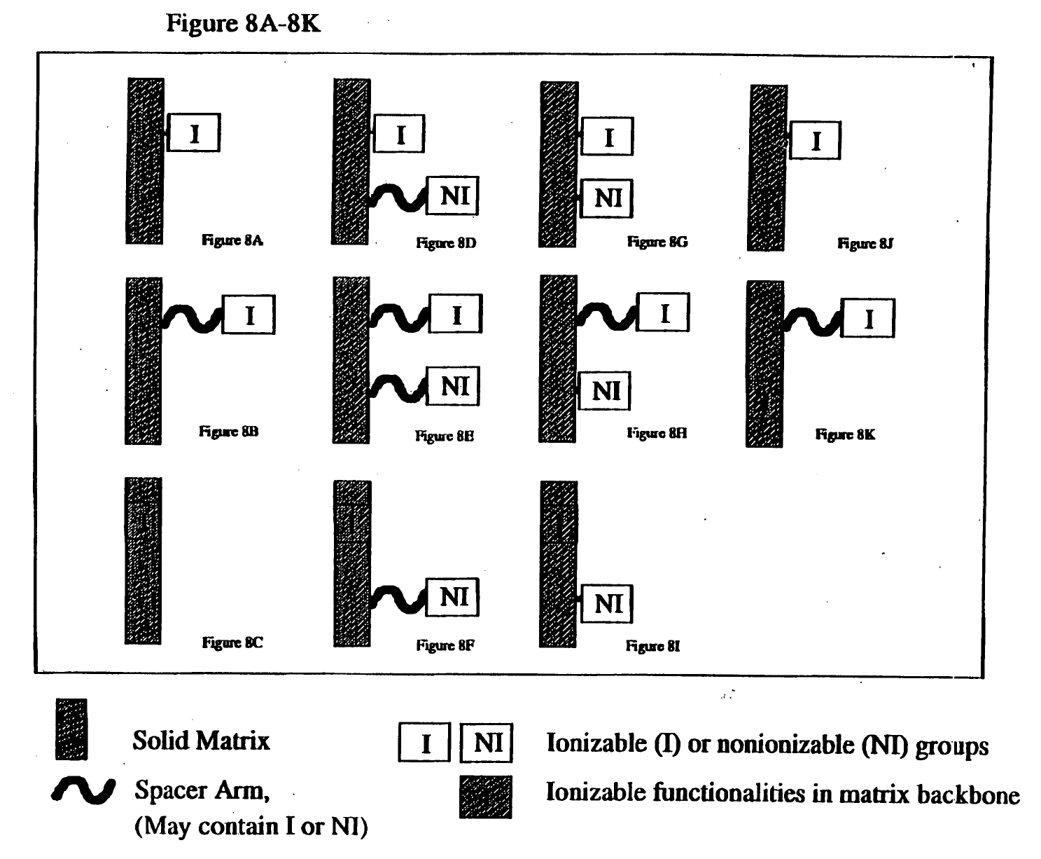

FIGS. 8A through 8K illustrate several configurations for the resins described herein. Specifically, FIG. 8A illustrates direct covalent attachment of the ligand to the solid support matrix which can be achieved by chemistry known per se in the art. For example, polymerization of acrylic acid will result in a solid support matrix having ionizable carboxyl groups directly and covalently linked to the matrix. Likewise, polymerization of acrylonitrile will result in a solid support matrix having non-ionizable groups directly and covalently linked to the matrix.

FIG. 8B illustrates covalent attachment of the ligand to the solid support matrix via a suitable spacer arm incorporated into and comprising part of the ligand which is well known in the art and is described in detail below. Suitable covalent attachment of the ligand to the solid support matrix includes covalent linkage through a thioether group, an ether group, an amide group, a urethane group, a disulfide group, a urea group, and the like. Spacer arms include, by way of example only, those derivatized from β-alanine, τ-aminobutyric acid (GABA), 6-aminocaproic acid, 1,6-diaminohexane, mercapto acids, histidine, tyrosine, and nitrotyrosine. Other suitable spacer arms are well documented in the art and the use of such spacer arms is not critical.

FIG. 8C illustrates a resin having ionizable functionality or a mixture of ionizable functionalities incorporated into the backbone of the solid support matrix.

FIGS. 8D-8K each illustrate further configurations for the resins described herein which can be achieved by chemistry known per se in the art. Unless otherwise indicated, the solid support matrix is non-ionizable.

The phrase “a solid support matrix having a selected ionizable functionality incorporated into the backbone thereof” as employed herein refers to solid support matrix containing within its backbone an ionizable functionality. Such ionizable functionalities are similar to ionizable ligands with the exception that the ionizable ligand is pendent to the backbone of the solid support matrix whereas the ionizable functionality is incorporated into the backbone. For example, such ionizable functionality can result from incorporation of a secondary or tertiary amino functionality in the backbone including, for instance, polyethylene amines which are well known in the art.

The term “electrostatically uncharged at the pH where the target protein or peptide is bound to the resin” means that less than 5% of the ionizable functionalities on the resin are charged at the pH of target protein binding. Preferably, less than about 1% of the ionizable functionalities on the resin are charged at this pH.

One skilled in the art recognizes that the degree of charge generated by an ionizable functionality depends upon the pH of the aqueous medium contacting the functionality and the pKa of the functionality. At the pKa of the resin, 50% of the ionizable functionalities are electrostatically charged and 50% of the ionizable functionalities are electrostatically uncharged. The adjustments in pH to achieve the requisite degree of electrostatically uncharged ionizable functionality on the resin is well within the skill of the art in view of the teachings herein.

The term “high ionic strength” means an ionic strength greater than or equal to that required to provide a conductivity of 4.7 millimho (milliSeimens (mS/cm²)). For example, such conductivity can be reached by using 250 millimolar (mM) sodium chloride. The term “low ionic strength” means an ionic strength less than 4.7 millimho. Procedures for determining the ionic strength of a solution are well known to those skilled in the art.

Methodology

The use of resins in the purification of proteins generally is well known in the art. These resins normally comprise a granular or beaded solid support matrix to which ligand is bound either directly thereto or through a spacer arm. A solution containing a target protein is contacted with the resin. Interactions between the resin and the target protein based on, for example, chemical charge, relative hydrophobicity, or specific affinity allow the proteins in the solution to bind to the resin. By specifically altering the conditions of the desorption buffer to counter the interactions between resin and protein, the target protein can be selectively desorbed.

The methods described herein represent an improvement over the protein recovery methods of the prior art insofar as these methods provide for a highly efficient means to recover industrial quantities of natural or recombinant protein from an aqueous medium such as a fermentation broth. In one particularly preferred embodiment, the methods described herein are particularly useful with proteins from a fermentation broth (e.g., chymosin and subtilisin) because purification may often be accomplished directly from a crude broth preparation and further because components of the crude clarified broth other than the target protein may not bind to these matrices.

The methods described herein are particularly useful where very high protein purity is not required. e.g., industrial enzymes, because this level of purity can be attained with one step purification. Optionally, broths can be loaded after pH adjustment, if required, without dilution, concentration, desalting, salt addition or particulate removal. It is contemplated that the resins described herein may have a regeneration advantage over resins which cannot be ionized.

The resins described herein provide a combination of several important features. They may allow direct recovery of a protein from crude broths and mixtures, and may allow significant purification at the start of downstream processing. The desalting effect may be well suited for a subsequent step in chromatographic processing, i.e., before ion exchange or affinity chromatography. They allow rapid, inexpensive desorption (e.g., elution) under non-harsh conditions. Minimal step or gradient technology is required, little or no extra salt is required, and little or no solvent is required.

Due to the general nature of the binding in the methods of this invention, these methods may exhibit less specificity than other procedures (e.g., chromatographic procedures). For example, different host organisms may have different major protein contaminant(s) with different hydrophobicities [11]. Therefore, a significant level of non-target protein binding may occur. Specificity using the methods described herein require that target protein hydrophobicity and/or isoelectric point (pI) differs from that of most contaminants. Proteins with similar hydrophobicity and isoelectric point as non-target protein contaminants may co-elute with the contaminants in the absence of gradient desorption. This can be countered by sample pretreatment or ligand, spacer and matrix variations, as described in detail herein. Additionally, since protein recovery is accomplished at either high or low ionic strength in the methods described herein, change of the ionic strength of the solution contacting the resin may remove some impurities while retaining the target proteins bound to the resin. Also, change of pH at low ionic strength may be useful in selectively desorbing one protein over another. On the other hand “total” protein binding is a preference in some processes, e.g., whey protein recovery. The methods described herein may also be useful for non-covalent protein immobilization [12-16], because binding is strong and protein recovery is simple.

The methods described herein below employ specific resins useful for the binding of target proteins. Binding to these resins is accomplished at a pH where the resin is electrostatically uncharged and is achieved principally by hydrophobic interactions. The degree of hydrophobic interaction between the resin and the target protein at the pH of binding can be rationally selected so that the strength of protein binding to the resin is controlled. Such selection is achieved by use of suitable matrices and ligands, including spacer arms, where the combination of these materials provides for controlled hydrophobicity of the resin.

One means to further control hydrophobicity is to include a population of non-ionizable ligands on the solid support matrix being either highly hydrophobic (e.g., containing phenyl or benzyl groups) or more hydrophilic (e.g., containing amide or urethane groups). Such factors are well within the skill of the art based on the teachings herein. Preferably, when non-ionizable ligands are employed, the population of non-ionizable ligands on the solid support matrix will range from 0 to 80 percent based on the total number of ligands (i.e., ionizable+non-ionizable ligands).

Additionally, when a ligand is attached to the solid support matrix through a thioether bond, the hydrophobicity of the resin may be controlled by oxidizing some or all of the sulfur atoms in the resin to sulfoxide and/or sulfone groups. By increasing the population of sulfoxide and/or sulfone groups present in the resin, the hydrophobicity of the resin is reduced. Suitable procedures for oxidizing thioether groups to sulfoxide or sulfone groups are well known in the art.

When an ionizable functionality is included on the solid support matrix backbone, it may not be necessary to include ionizable ligands on this matrix and, when such are not employed, all of the ligands, if any, attached to the solid support matrix will be non-ionizable. In this embodiment, adjustment of the quantity and type of non-ionizable ligands, attached to the solid support matrix permits further control of the resin's hydrophobicity.

The methods described herein are useful for recovering a broad range of proteins including recombinant products. The methods may also be used to perform protein separations on extracts from natural sources, such as plant and animal sources.

The resins described herein employ ionizable and/or non-ionizable ligands attached to solid support matrices. Any method of covalent attachment of the ligands may be used, provided that the attachment does not result in introduction of ionizable groups other than the desired ionizable group on the ligand. Examples of methods for attachment of such ligands to the solid support matrices include covalent thioether, ether, amide, and urethane bonds. Other thioether methods, disulfide attachment, and urea methods may also be used. Additionally, hydrazide ligands may be attached to epoxide or aldehyde functional groups. Representative ligand attachment chemistries are shown in Table 1. Other ligand attachment chemistries are illustrated elsewhere herein and/or are known in the art.

TABLE 1
Ligand Attachment Chemistries

| Matrix Functional | | Ligand | |
|---|---|---|---|
| Group | Activation Chemistry | Reactive Group | Linkage |
| Hydroxyl | carbonyl diimidazole | amine | urethane |
| | epoxide | amine | amine |
| | | thiol | thioether |
| | | hydroxyl | ether |
| | CNBr | amine | various |
| | tosyl chloride | amine | amine |
| | | thiol | thioether |
| | divinylsulfone | amine | amine |
| | | thiol | thioether |
| | | hydroxyl | ether |
| Carboxyl | carbodiimide | amine | amide |
| Amine | carbodiimide | carboxyl | amide |
| Amide | hydrazine/HNO₂ | amine | amide |

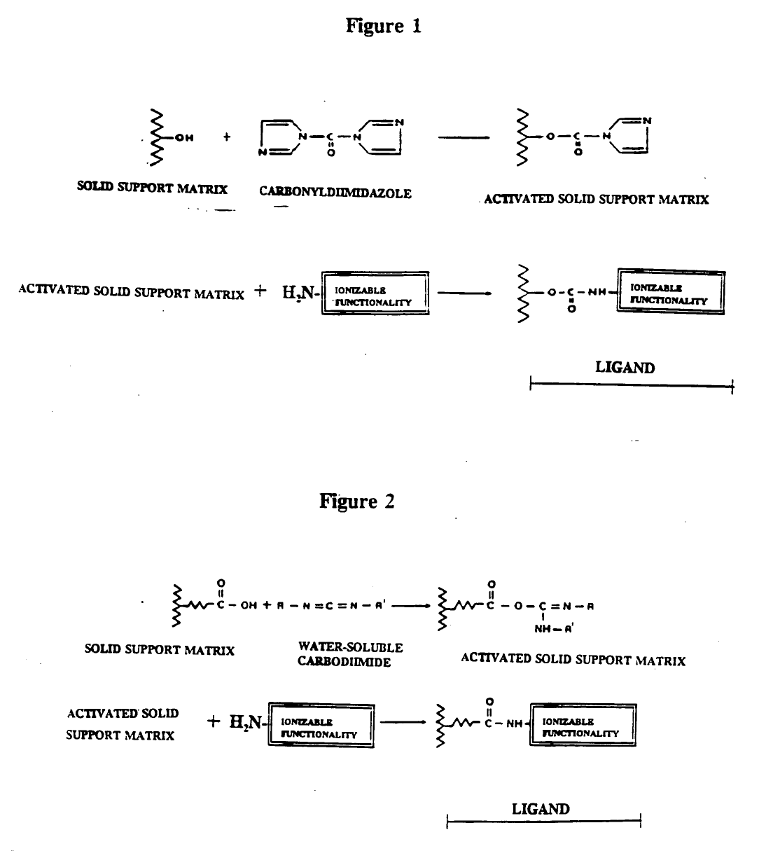

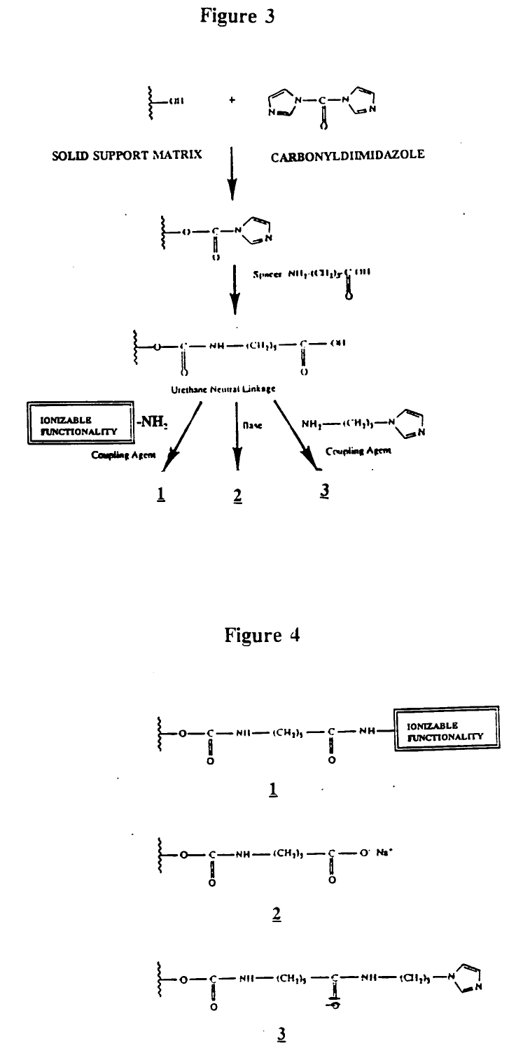

Each of these chemistries is well known in the art and coupling results in the linkage described above. For example, the ligands may be attached to the solid support matrix such as activated cellulose matrix through a neutral urethane bond by use of carbonyl diimidazole (CDI) reagent as illustrated in FIGS. 1 and 3. Alternatively, the ligands may be attached through an amide bond to a cellulose matrix derivatized with an aminocaproic acid spacer arm as illustrated in FIG. 4, embodiments 1 and 3. Where necessary, 100% substitution of spacer arm carboxyl groups can be achieved. Such substitution can be confirmed using procedures well known in the art, such as small ion titration.

Ligand attachment via carbonyl diimidazole (CDI) activated matrices is illustrated in FIG. 1. Carbodiimide coupling of the ligand to matrix carboxyl groups is illustrated in FIG. 2. CDI-caproic acid supports are illustrated in FIG. 3. Possible derivations of the CDI-caproic matrices are illustrated in FIG. 4.

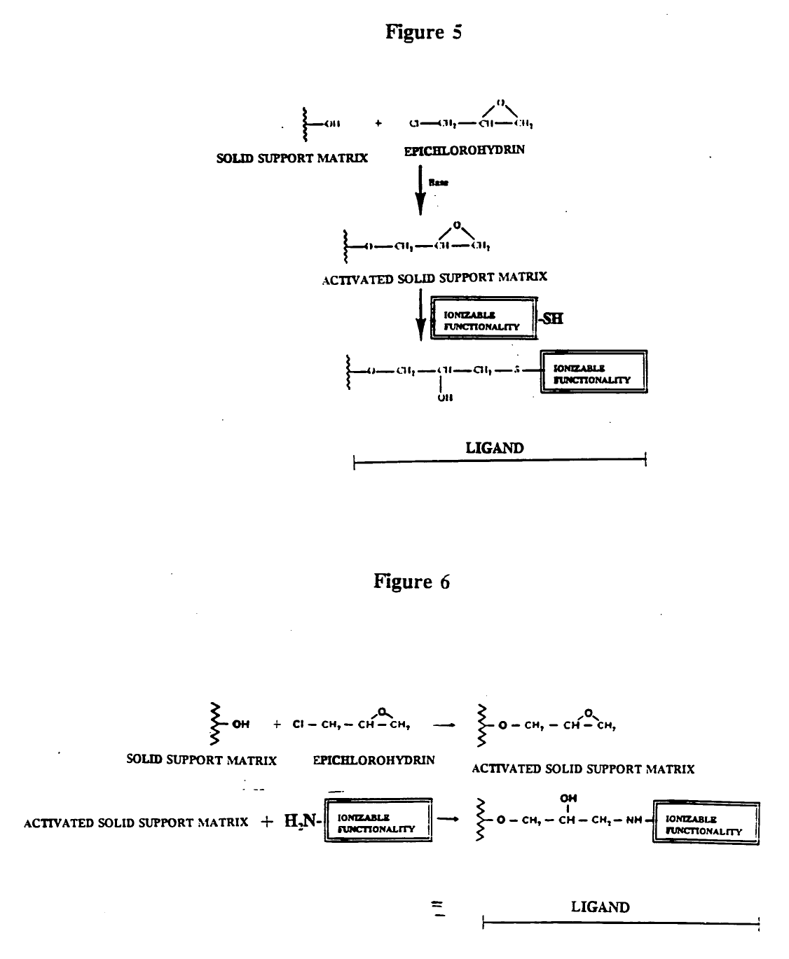

Alternative to CDI activated matrices for ligand attachment are resins containing epoxide groups whose preparation and ligand attachment thereto are exemplified in FIGS. 5 and 6. Specifically, FIG. 5 illustrates attachment of thiol containing ligands to an epoxide activated solid support matrix. In this resin, the ligand is attached through a stable, neutral thioether bond [10]. Ligands which can be used with this chemistry comprise functional groups which include, by way of example, mercaptobenzimidazole. 4-mercaptopyridine, 2-mercaptopyridine, methimazole and 4-hydroxythiophenol. FIG. 6 illustrates the reaction of an amine with an epoxide activated solid support matrix. The chemistry used in both cases is typically aqueous and therefore less expensive than the CDI method. However, this chemistry can introduce cross-linking into the matrix which may reduce loading capacity. Additionally, activation levels using this chemistry are typically low.

Other preferred activation agents for matrix modification with thiol ligands have one highly reactive group and one less reactive group. The highly reactive group reacts with a matrix hydroxyl group at alkaline pH to form a stable ether bond, while the second group does not react under these conditions. This prevents cross-linking. After the first activation step, the second group may be reacted directly with a radical reagent or a strong nucleophile, e.g., a thiol. The second group may also be modified to a more reactive form, then reacted with the nucleophile. Allyl halides (e.g., allyl bromide) and allyl glycidyl ether are preferred reagents. For example, solid support matrices activated with allyl glycidyl ether can permit substitution levels greater than those activated with epichlorohydrin. Upon covalent attachment of these ligands to the resin, the allyl group can be modified so as to incorporate an ionizable functionality (e.g., reaction with bromine water to form the bromo derivative followed by reaction with the bis sodium salt of 4-hydroxybenzoic acid).

It is understood, however, that the ionizable ligand may be directly attached to the resin. Under these circumstances, the resin is prepared to include direct attachment of the ionizable ligand or can be derivatized to include such direct attachment. For example, polymerization of a monomer composition comprising methyl acrylate will provide for a polymer comprising methyl acrylate units which can be derivatized by solvolysis to provide for acrylic acid units in the polymer.

It is also understood that ionizable functionality can be incorporated into the solid support matrix. For example, a polyamine or a polymer comprising amine functionality can be used wherein the amine functional groups are ionizable at low pHs. In such an embodiment, the polymer can be prepared to include other functional groups and ligand attachment can be made either through the amine groups or such other functional groups. In the former embodiment, ligand attachment chemistries are employed to retain at least a portion of the ionizable functionality in the backbone of the matrix. When ionizable functionalities are included in the backbone, it is not necessary for the ligands to contain ionizable functionalities and in one embodiment the ligands attached to the matrix are non-ionizable and in another embodiment, at least a portion of the ligands are ionizable.

Ionizable ligands useful on the resins described herein include those derived by covalently linking 3-(aminomethyl)pyridine (3AMP), 4-(aminomethyl)pyridine (4AMP), 1-(3-aminopropyl)imidazole (API), 2-(aminomethyl)benzimidazole (AMB), 4-(3-aminopropyl)morpholine (APM), histamine, and the like to the solid support matrix employing the chemistry described above in Table 1 and in the Examples herein below. Such attachment chemistry includes CDI activated and CDI-caproic acid activated cellulose which are described above and in the figures attached hereto.

When a hydrophobic amine ligand is used, it is preferably selected from the group consisting of 2-phenylethylamine, L-phenylalaninol, (1R, ²S)-(−)-phenylpropanolamine, tryptamine, 4-(2-aminoethyl)-benzenesulfonamide, (1S, 2S)-(+)-2-aminophenylpropanediol and I-hexylamine.

Other useful ligands include those containing unsubstituted phenol and substituted phenols with pKa's in the 6-9 range. These may be useful for the negatively ionizable ligand type. Suitable substituents for the phenyl group include nitro, halo (e.g., chloro, bromo), alkyl of from 1-10 carbon atoms, alkoxy of from 1-10 carbon atoms, carboxyl esters where the ester group is from 1 to 10 carbon atoms, cyano, carbonyl alkyl (—C(O)R) groups of from 1-10 carbon atoms, and mixtures thereof. Typically, substituted phenyl groups have from 1 to 4 such substituents and preferably from 1 to 2. The above substituents can also be attached to other substitutable ligands including ligands containing pyridyl groups, histidyl groups, indolyl groups, imidazolyl groups, morpholinyl groups, benzimidazolyl groups and the like.

The list of ionizable ligands set forth herein is not intended to be exhaustive nor is the chemistry employed to covalently attach these ligands to the solid support matrix. Suffice it to note that suitable ligands are well known in the art as well as the chemistry employed for covalent bonding of these ligands to the matrix. Suffice it to further note that common to the ionizable ligands as well as any ionizable functionality in the solid support matrix is the presence of one or more functional groups which can be electrostatically charged at one pH and electrostatically uncharged at another pH. The particular functional group employed on the resin is not critical.

The ionizable ligand and/or ionizable functionality is preferably present on the resin at a sufficient concentration to permit target protein binding at both high and low ionic strength. Preferably, the ionizable functionality will be present on the resin at a concentration of 0.4 mmol to about 3 mmol per gram dry weight of resin (or 0.05-0.5 mmol/ml). In one particularly preferred embodiment, non-ionizable ligands are employed in conjunction with ionizable functionality so as to provide for further control over the degree of hydrophobicity/hydrophilicity at the pH of target protein binding to and desorption from the resin. In this embodiment, the percentage of non-ionizable ligands relative to the total number of ligands ranges from about 0 to about 80 percent, preferably from 0 to 40 percent.

In the methods of this invention, a solution or aqueous medium comprising the target protein to be recovered is contacted with the resin at a pH wherein the resin is electrostatically uncharged. At this pH, binding is primarily by hydrophobic interaction and the degree of hydrophobicity of the resin can be adjusted by modification of any spacer arms employed to attach the ligand to the resin, by employing a more or less hydrophobic solid support matrix, by use of non-ionizable ligands, by the use of a more or less hydrophobic ionizable ligand, by adjusting the density of ligands on the matrix and combinations thereof. In view of the teachings herein, one skilled in the art can rationally adjust these parameters based on the properties of the target protein coupled with the enhancements desired.

The medium comprising the target protein, e.g., chymosin, to be recovered may be derived from any known protein sources, including microbial or animal sources. For example, chymosin solutions can include fermentation broth from Aspergillus, E. coli, yeast, as well as aqueous extracts obtained from bovine stomachs.

The aqueous medium may include a buffer and/or a salt to enhance binding efficiencies to the resin. Suitable salts are those conventionally employed in protein chromatography and include, by way of example, the lithium, sodium, potassium and ammonium salts of chloride, sulfate, phosphate and acetate. Preferably, sodium chloride is employed because it is effective, inexpensive and safe. As noted above, the resins described herein will bind protein from aqueous media at both high and low salt concentrations. Specifically, these resins will bind about 50 percent or more of the target protein in the aqueous medium at low and high salt concentrations. It being understood, of course, that the resin employed has sufficient capacity to bind all of the target protein in the medium.

The aqueous medium is contacted with the resin for a time sufficient to allow the target protein to bind to the resin. This contact may be made, for example, where the resin is packed in a column, employed in a fluidized bed, or suspended in a stirred batch system where the resin is mixed with the aqueous protein medium. Under such conditions, the target protein binds to the resin thereby forming a resin-protein complex. After contacting the aqueous medium with the resin the resin is then washed with a buffer of pH equal to that of the aqueous medium to separate the aqueous medium from the resin and the proteins bound thereon. This buffer may additionally comprise up to at least 2 M salt as listed above.

The target protein is then desorbed from the resin merely by contacting the resin with an aqueous medium having a pH which induces an electrostatic charge on the resin. The electrostatic charge induced on the resin may be of the same or different, i.e. opposite, polarity from that of the target protein. In a preferred embodiment of the present invention, the induced charge on the resin is of the same polarity as the net electrostatic charge on the target protein at the pH of desorption and the resulting charge-charge repulsions between the resin and the target protein being sufficient to overcome any hydrophobic interactions with the resin thereby facilitating desorption from the resin. This can be achieved by rationally selecting the relative hydrophobicity of the resin as described above and/or by incorporation of sufficient ionizable ligands to provide for an effectively large electrostatic charge on the resin at the pH of desorption.

In another preferred embodiment of the present invention, the induced charge on the resin is of the opposite polarity from the net electrostatic charge on the target protein at the pH of desorption. In this embodiment, desorption of the target protein from the resin can be facilitated by, for example, use of a desorbing solution of high ionic strength. In either embodiment, the use of a polarity reducing agent, such as propylene glycol, can facilitate desorption of the target protein or peptide from the resin.

Ligand choice is based on protein pH constraints. For example, in certain circumstances, it may be desirable to use a resin having an inducible positive charge at pHs where the target protein is stable so that extreme pHs can be avoided for protein binding or desorption. Likewise, a ligand with an inducible negative charge at pHs where the protein is stable may be preferred. Such ligands include, by way of example, caproic acid (titrates from pH=3.3). Phenolic ligands, e.g., tyramine, are also useful in this regard since phenol titrates from about pH 6 and above. Nitrated or chlorinated phenol or tyramine ligands are especially useful since they have a pKa value near neutral pH.

In another embodiment where the protein is stable only at physiological pHs, the ionizable ligands attached to the solid support matrix should provide resins which begin to titrate (become electrostatically charged) in a pH range of from about 5 to 9 and more preferably from about 5.5 to about 8.5. Such a range permits binding and desorption of the target protein at a pH range which, in the case of many proteins, reduces risks of denaturation, etc. as compared to more extreme pH ranges.

In the methods described herein, ligand density on the matrix is selected so that target protein binding can be achieved at high and low ionic strength. This permits processing of the aqueous protein media without dilution, concentration, desalting, salt addition or particulate removal. Another advantage contemplated by the use of the resins described herein is that these resins may suffer less fouling than matrices previously employed for separating proteins from broths.

Upon separation of the target protein from the resin in the manner described above, the recovered target protein solution can be further treated by conventional methods.

The resin employed in this process can be regenerated by conventional methods. For example, resins having an inducible positive charge may be regenerated using 0.1 M HCl, with or without a polarity reducing agent such as ethanol or ethylene glycol. Likewise, resins having an inducible negative charge may be regenerated using 0.1 M NaOH, again with or without a polarity reducing agent such as ethanol or ethylene glycol. However, this latter process can result in hydrolysis of CDI matrices and can cause matrix swelling. Cross-linking the cellulose matrix before activation has been used to reduce swelling and improve column flow rates. Cross-linking may also be used with positively ionizable matrices.

When foulant is a problem, pretreatment of broth or other crude media by flow through an inexpensive resin such as DEAE at high ionic strength may improve separation. For example, use of pretreatment with a DEAE column with subtilisin samples results in significant depletion of foulants, with more than 99% enzyme activity remaining. Depletion of foulants improves regeneration, resin lifetime, and capacity. This extra step may be especially feasible where the flow through from the pretreatment column can be loaded directly onto the separation systems of the present invention without buffer adjustment.

In one example of the methods described herein, chymosin bound to CDI-caproic acid cellulose (or Sepharose) at pH 2 where the carboxyl group is essentially electrostatically uncharged. Elution was at pH 6 where the carboxyl group is essentially charged and desorption involves a charge-charge repulsion between the resin and the chymosin.

In a second example, resins using pyridine/imidazole functionalities are useful for purifying some proteins. For example, cellulose-CDI-caproic acid was 100% substituted with 3-diethylaminopropylamine (DEAPA) (pKa≈9.5) and 1-(3-aminopropyl)imidazole (API) (pKa≈6.2). These resins do not bind proteins such as chymosin at 1 M NaCl, pH 5.5, where these resins are largely charged at this pH. Without being limited to any theory, it appears that the positive charges on these resins disrupt hydrophobic binding. Contrarily, chymosin successfully binds at 0.5 M NaCl, pH 6, to a resin prepared by reacting CDI-activated Perloza with 2-(aminomethyl)pyridine (pKa≈4.1) at which pH value the resin is largely electrostatically uncharged. The pyridyl resin is eluted with a pH 2 buffer which titrates resin pyridine groups to the charged form. Note the preferred working pH range for chymosin is 2 and 4.5-6.5 and the pyridyl resin satisfactorily works in this range.

The above results illustrate the use of a compatible resin in conjunction with the target protein to effect recovery. Accordingly, the buffer systems and hence the ionizable functionalities used for protein recovery are dependent on the target protein to be purified. For example, unlike chymosin, subtilisin is unstable below pH 4.5, so buffers below that pH cannot be used. In addition, citrate buffers should be avoided when working with subtilisin in order to avoid Ca²⁺ chelation, since Ca²⁺ stabilizes the enzyme. Additionally, phosphate buffers can precipitate Ca²⁺ and, therefore, such buffers should also be avoided. The preferred working range for subtilisin is pH 5-7. Operation in the range of pH 7-10 may be acceptable for limited periods.

With subtilisin recovery methods, buffers with molarities of from 100-200 mM are preferred for the initial pH adjustment required for desorption. Once pH is adjusted, buffer molarity can be reduced by dilution. Acetate buffer (pH 5.2) gave efficient recovery for most hydrophobic positively ionizable matrices. A glycol buffer containing 8% formate, 40% propylene glycol, pH 5.5 desorbed subtilisin from all such matrices tested. With hydrophobic negatively ionizable matrices, desorption efficiency was enhanced by increasing pH, but subtilisin stability was reduced. The glycol buffer at pH 7-9 was preferred. This gave rapid pH adjustment and good desorption profiles without extremes of pH.

In another example, hydrophobic amine ligands were used. These ligands were attached to epoxy Sepharose and are largely electrostatically uncharged at pH 10. At pH values below 10, these matrices become electrostatically charged at the secondary amine linkage. Loading at pH 10.5, at low ionic strength, a subtilisin varient (as described in U.S. Pat. No. 5,185,258, issued Feb. 9, 1993 and incorporated herein by reference in its entirety) was bound using the following ligands: APP, AEBS, and tryptamine. Only tryptamine Sepharose bound at pH 10.5+0.5 M NaCl. None of the matrices bound strongly at pH 9, although subtilisin passage through these matrices was retarded. The above indicates that a strongly hydrophobic ligand and extreme pH were required for subtilisin purification using such resins.

The high pH required for loading of subtilisin is unsatisfactory because of stability problems. Therefore, resins that are electrostatically uncharged at pH 7-9 and which would allow subtilisin binding at these pH's, but would ionize partially or wholly at pH 5-6, causing subtilisin desorption were developed. One example of such resins is a 100% API-substituted CDI-caproic cellulose which is electrostatically uncharged above pH 8 (at high ionic strength) and binds subtilisin bound at pH 8.5.

Another example was a mixed resin of APP (67%) and API (33%) which binds subtilisin at pH 8.0.

Still other examples include resins containing 100% substituted 3- and 4-pyridyl ligands which are electrostatically uncharged above pH 6.5 and bind subtilisin at pH 7.

Target protein may be desorbed from each of these resins by a reduction in pH of the buffer contacting the resin. For example, subtilisin was desorbed from each of these materials by a pH reduction to 5.2, which titrated some of these ionizable ligand groups to the protonated form. Therefore, binding of subtilisin was by hydrophobic interactions, with the possibility of a contribution from hydrogen bonding and charge transfer, and desorption was by charge repulsion and/or disruption of hydrophobic interactions. These resins may be loaded at high or low ionic strength, thus removing the requirement for dialysis or dilution in sample pretreatment.

In the case of chymosin, desorption of this protein from the resin uses a buffer solution having pH adjusted to below about pH 4, preferably pH 2. A preferred desorption buffer solution additionally comprises from about 20 mM to about 50 mM potassium or sodium chloride.

A further embodiment of the methods described herein relates to methods of purifying proteins, including enzymes, utilizing the above described resins. It is understood that the target protein may be in an aqueous medium containing other proteins from which it is to be purified. This medium may be crude fermentation broth which includes, in addition to proteins, a wide variety of biologicals, including amino acids, polysaccarides, sugars, organic acids and salts. These methods are described in detail herein using chymosin and subtilisin as examples, although it is contemplated that other target proteins may be purified using the disclosed resins and methods.

In a particular embodiment, the present invention relates to a method for separating chymosin from an aqueous medium of proteins comprising chymosin which method comprises contacting the aqueous medium of proteins with any of the previously described resins for a sufficient time to allow the chymosin to bind to the resin; separating the resin/chymosin complex from the aqueous medium; then recovering chymosin from the resin. The chymosin to be purified may be derived from any of the known enzyme sources, including Aspergiilus. E. coli, yeast, and bovine stomachs. A similar process for subtilisin separation and purification is also encompassed within the present invention.

In the process embodiment where the resin comprises a positively inducible ligand bound to a solid support matrix and the target protein comprises chymosin, the method preferably comprises adjusting the pH of the aqueous medium such that the resin is electrostatically uncharged. This method may further comprise adding up to 2 M salt to the aqueous medium of proteins prior to contacting it with the resin. Useful salts include those listed above.

The aqueous medium of proteins is then contacted with the resin for a time sufficient to allow the chymosin to bind the resin. This contact may be made, for example, where the resin is packed in a column, employed in a fluidized bed, or suspended in a stirred batch system where the resin is mixed with the aqueous protein medium, then filtered away from the aqueous medium. After contacting the aqueous medium with the resin, the resin is then washed with a buffer of pH equal to that of the aqueous medium to separate the aqueous medium from the resin and the proteins bound thereon. This buffer may additionally comprise up to at least 2 M salt as listed above.

The chymosin can then be recovered from the resin using a buffer solution having pH adjusted sufficiently low as to induce a positive charge on the resin. A preferred desorption buffer solution additionally comprises from about 20 mM to about 50 mM sodium or potassium chloride.

Although the resins described herein have particular utility for large scale protein recovery using simple binding and desorption procedures, the resins and methods described herein may be used for FPLC and HPLC analytical or high value preparative use. In particular, descending salt gradients may be used with electrostatically uncharged matrices and (a) pH shift to the electrostatically charged form followed by an increasing salt gradient or (b) pH gradient elution to further shift away from the pH of the neutral form.

By using a mixture of affinity ligands plus ionizable ligands, the advantages of affinity binding may be coupled with facile desorption by electrostatic repulsion. Binding would be at a pH where the titratable ligand is electrostatically uncharged or inert and desorption would be by pH change.

Further, the resins and systems of the present invention may be applied to non-chromatography resin systems such as liquid-liquid extractions and polymer/UF systems. Modified phase-separating polymers, such as polyethylene glycol, for liquid-liquid extraction [19,20], modified membranes [21] or soluble polymer-UF methods [22,23] may also employ the systems of the present invention. In such embodiments, the resin and the matrix need not be solid or water insoluble.

### EXAMPLES

The following examples are presented to illustrate specific embodiments of the present invention and should not be interpreted as limitations upon the scope of the invention.

Examples I to VI demonstrate methods for preparing activated resins and/or subsequent attachment of representative ligands. Example VII illustrates the binding capacity of representative resins for the enzyme subtilisin. Examples VIII and 1×show typical titration data for representative resins useful in the present invention. And in Examples X and XI, recovery of subtilisin is demonstrated.

In these examples, the abbreviations used have the following meanings. If not defined, any abbreviation used below has its generally accepted meaning.

| | |
|---|---|
| AEBS = | p-(2-aminoethyl)benzenesulfonamide; |
| API = | APImidazole = 1-(3-aminopropyl)imidazole; |
| AMB = | AMBenzimidazole = 2-(aminomethyl)benzimidazole; |
| APM = | APMorpholine = 1-(3-aminopropyl)morpholine; |
| 2AMP = | 2AMPyridine = 2-(aminomethyl)pyridine; |
| 3AMP = | 3AMPyridine = 3-(aminomethyl)pyridine; |
| 4AMP = | 4AMPyridine = 4-(aminomethyl)pyridine; |
| APP = | (1S, 2S)-(+)-2-aminophenylpropanediol: |
| CDI = | carbonyl diimidazole; |
| CM = | carboxymethyl; |
| CMC = | 1-cyclohexyl-3-(2-morpholinoethyl)carbodiimide; |
| CV = | column volume; |
| DAH = | diaminohexane; |
| DEAPA = | 3-diethylaminopropylamine; |
| DMF = | dimethylformamide; |
| DMSO = | dimethylsulfoxide; |
| ECH = | epichlorohydrin; |
| EDC = | (1-ethyl-3-(3-dimethylaminopropyl)carbodiimide; |
| EEDQ = | N-ethoxycarbonyl-2-ethoxydihydroquinoline; |
| g = | grams; |
| HEX = | hexylamine = 1-hexylamine; |
| LIS = | low ionic strength; |
| M = | molar; |
| mg = | milligrams; |
| MG1 = | 1-(3-aminopropyl)imidazole-substituted Perloza resin; |
| MG2 = | 4-(aminomethyl)pyridine-substituted Perloza resin; |
| mM = | millimolar; |
| mmho = | millimho (milliSeimens/cm² or mS/cm²); |
| mmol = | millimoles; |
| MB = | mercaptobenzimidazole; |
| methimazole = | 2-mercapto-1-methylimidazole; |
| MP = | 4MP = 4-mercaptopyridine; |
| P = | Perloza; |
| PCC = | Perloza CDI aminocaproic acid resin; |
| PCDI = | CDI activated Perloza; |
| PPA = | phenylpropanolamine; |
| S = | Sepharose; |
| TRPN = | tryptamine. |

Using the above abbreviations, the resins employed in the following examples are generally abbreviated as follows: [Matrix]-[Coupling Method]-[Spacer Arm, if any]/[Ionizable Functionality]. For example, “P CDI 4AMPyridine” refers to a Perloza solid support matrix (P) activated with carbonyl diimidazole (CDI) and coupled with a 4-aminomethylpyridine group; “S ECH MB” refers to a Sepharose solid support matrix (S) activated with epichlorohydrin (ECH) and coupled with a mercaptobenzimidazole (MB) group; and “P CDI DAH Hydroxyphenylacetic acid” refers to a Perloza solid support matrix (P) activated with carbonyl diimidazole (CDI) and coupled through a diaminohexane spacer (DAH) with hydroxyphenylacetic acid. It being understood, of course, that the spacer arm (if any) and the ionizable functionality(ies) comprise the ionizable ligand.

### Example I

### Epoxide Activation

Sepharose 6B (50 g), previously washed with 10 volumes of water, was mixed with 47 ml of 1 M NaOH and 5 ml of epichlorohydrin for 24 hours at 4° C. Epoxide group substitution was determined by methods known in the art to be 1.06 mmol/g. Similar activations gave epoxide group substitutions of 1.28 and 1.02 mmol/g.

### Example II

### Amine Ligand Attachment to Epoxidated Sepharose

Epoxidated Sepharose (10 g, 1.06 mmol/g dry) prepared as described in EXAMPLE I was mixed with a 5 molar excess (i.e., 5 moles of ligand/mole of resin reactive groups) of a ligand selected from AEBS, APP, or TRPN solvated with 4 ml DMSO and 2 ml water for 24 hours at 37° C. A 5 molar excess of HEX was mixed likewise with 10 g epoxidated Sepharose (1.28 mmol/g dry). The resulting resins were washed with 10 volumes of 50% DMSO (DMSO:water, 1:1) (tryptamine resin only), 10 volumes of water, 2 volumes of 0.1M HCl and an additional 10 volumes of water. Ligand substitution was determined by 0.1 M HCl titration to pH 4 to be 0.81 mmol/g for APP Sepharose and 0.99 mmol/g for HEX Sepharose. This represents approximately 80% efficiency of ligand substitution of epoxide groups.

### Example III

### Allyl Glycidyl Ether Activation

Perloza cellulose (Perloza MT 100 fine beaded cellulose available from Secheza, Czechoslovakia) was washed with 5 volumes of water (MilliQ grade water) and 3 volumes of 0.3 M NaOH and suction dried. A 40 g quantity of the matrix was mixed with 12 ml of 99+% allyl glycidyl ether by vigorous shaking. The mixture was left at room temperature for 48 hours with occasional shaking. The activated matrix was washed with 10 volumes of water and suspended in 3 volumes of water. Bromine water (1%) was added slowly over 5 minutes until the mixture no longer decolorized the bromine water. The brominated resin was washed with 10 volumes of water. Allyl group concentration on the resin was determined by the amount of bromine water decolorized. The concentration of reactive bromine groups on the resin (1 g sample suspended in 9 ml of water) was determined by substitution with 0.5 g sodium sulfite (4 hours, 60° C.), followed by 0.1 M NaOH titration to pH 8.

### Example IV

### Thiol Ligand Attachment

A resin (5 g), produced as described in EXAMPLES I or III above, was suspended in 5 mL of 1 M phosphate buffer (pH 7) and flushed with nitrogen. A 5 molar excess of a ligand selected from MB, 4 MP or methimazole, dissolved in 5 ml of DMSO, and 0.1 g of sodium borohydride were added and the mixture was reacted for 6 hours. Resins produced by the method of EXAMPLE I were maintained at room temperature. Resins produced by the method of EXAMPLE III were maintained at 60° C. The resulting thioether resin was washed with 5 volumes of 0.1 M HCl, 10 volumes of water, 5 volumes of 0.1 M NaOH and 20 volumes of water. A sample (1 g) was titrated with 0.1 M HCl to pH 3.5-2.7 (lower end pH for MB ligand).

### Example V

### CDI Activation and Ligand/Spacer Arm Attachment

Sepharose CL6B and Perloza cellulose were activated with CDI and titrated by methods known in the art. Activation levels between 1.0 and 3.5 mmol/g were obtained using 20 to 80 mg CDI per g Sepharose and 30 to 120 mg CDI per g cellulose.

Amine reagents soluble in dioxane API, APM, histamine, AMB, 4AMP, 2AMP, DAH, tyramine (prepared from the hydrochloride by dissolving in water, adjusting the pH to 12 with 1 M NaOH and freeze-drying) and dibromotyramine (prepared from tyramine hydrochloride by methods known in the art)) were reacted directly with 10 g samples of dioxane solvated CDI activated matrices (1 ml of water was included with the tyramine ligands). A 5 molar excess of amine reagent was used except for DAH which was used in 10 molar excess. Dioxane (5 ml) was added and the resin was mixed for 24 hours at room temperature. The resins were washed with 3 volumes of 75% dioxane, 2 volumes of 33% dioxane, 10 volumes of water. 2 volumes of 0.1 M HCl and a further 10 volumes of water.

Aminocaproic acid and nitrotyrosine (sodium salt forms) were not soluble in dioxane. Therefore, a 40% solution of sodium aminocaproate was prepared by dissolving 80 g of aminocaproic acid in 64 ml of 10 M NaOH and water to a final volume of 200 ml. Dioxane solvated activated cellulose (300 g resin+150 ml dioxane) and Sepharose (100 g resin+50 ml dioxane) were mixed with 80 ml and 30 ml respectively of the sodium aminocaproate solution for 24 hours at room temperature. The aminocaproic resins were washed with 2 volumes of 66% dioxane, 2 volumes of 33% dioxane and 20 volumes of water. Similarly, nitrotrosine was dissolved in water and 3 M NaOH to produce a 15% solution at pH 11.3. Activated cellulose (10 g), solvated with 50% dioxane was mixed with 13 ml of nitrotyrosine solution for 24 hours at room temperature.

### Example VI

### Ligand Attachment By Amide Bonds

A. Coupling of Amine Ligands to Resin Carboxyl Groups

A 5 molar excess of a ligand selected from 4AMP, 3AMP, DEAPA or API was adjusted to pH 4.7 with 6 M HCl and mixed with aminocaproic resin (1.55 mmol/g) prepared as described in EXAMPLE V. The pH was adjusted to 4.7 with 1 M HCl or NaOH as appropriate and a 3 molar excess of water soluble carbodimiide (EDC, 50% solution in water) was added. The pH was maintained at 4.7 for 1 hour with 1 M HCl or NaOH as appropriate and mixed for a further 10 hours without adjustment at room temperature. A further 1 molar excess of EDC was added and the pH adjusted for 1 hour and stirring continued for 10 hours as before. The resin was then washed with 10 volumes of water, 2 volumes of 0.1 m HCl and 10 volumes of water. Ethanolamine was coupled to nitrotryrosine cellulose by the same method. No residual carboxyl groups could be detected on these resins by titration methods sensitive to 20 micromol/g.

B. Coupling of Carboxyl Ligands to Diaminohexane Resins

The method used was similar to that of EXAMPLE VI(a) above but the pH of the ligand solution (containing a ligand selected from 4-hydroxyphenylacetic acid, 3-chloro-4-hydroxyphenylacetic acid or 3-nitro-4-hydroxybenzoic acid) was adjusted with 1 M NaOH, the pH was maintained at 5, diaminohexane cellulose was used and a 0.1 M NaOH wash replaced the HCl wash.

For dichlorosalicylic acid, a sodium salt was prepared by addition of 1 M NaOH to a suspension of the acid (0.3 g) in water until the pH was stable at 7 and the solution was then freeze dried. The resulting salt was dissolved in 10 ml of DMSO and mixed with EEDQ (0.5 g dissolved in 5 ml of ethanol) and 50% DMSO solvated diaminohexane cellulose. The mixture was shaken for 6 hours at room temperature. A further 0.5 g of EEDQ was added and the reaction continued for 18 hours. The resin was washed with 5 volumes of DMSO, 2 volumes of 50% DMSO, 10 volumes of 0.1 M NaOH and 10 volumes of water. Coupling of resin amine groups by these methods was only 80 to 90% complete. Residual amino groups were blocked by methods known in the art (e.g., by acetylation).

### Example VII

### Resin Capacity for Crude Subtilisin

Capacity testing was conducted by batch operation using a subtilisin varient obtained as described in U.S. Pat. No. 5,185,258, issued Feb. 9, 1993, the entire disclosure of which is incorporated herein by reference. Load buffers used were:

pH 8: 25 mM TRIS™+0.5 M NaCl

pH 7: 10 mM phosphate+0.5 M NaCl

The resin was equilibrated with load buffer (5 volumes) and suction dried. One sample of the resin (2 to 4 g) was weighed into a 10 ml measuring cylinder, suspended in load buffer and left to settle for 48 hours (to determine the resin weight:volume ratio). Another sample of resin (0.1 to 0.15 g) was weighed into a 25 ml vial and mixed with 8 ml of subtilisin previously adjusted to load pH with 1 M NaOH for 1 hour at 4° C. on a rotating wheel. The contents of the vial were quantitatively transferred to a Pierce disposable 2 ml minicolumn and washed with 10 ml of load buffer. The resin was eluted with 25 mM acetate buffer (pH 5.2) into a 10 ml volumetric flask. The elution was assayed for enzyme activity (using the succinyl-ala-ala-pro-phe-pNA substrate assay method) and protein content (using absorbance at 280 nm and the bicinchonic acid assay). Capacity data, expressed in mg protein/ml of resin, is shown in Table 2. Activity refers to the concentration of subtilisin calculated to provide the active protein concentration in mg/ml.

TABLE 2
Subtilisin Capacity Data

| | Batch Capacity mg/ml | | |
|---|---|---|---|
| | | Absorbance | |
| Resin | Activity | (280 nm)* | Protein** |
| PCC 3AMPyridine | 13 | 51 | 50 |
| Load pH 7.0 | | | |
| PCC 4AMPyridine | 15.2 | 59 | 35 |
| Load pH 7.0 | | | |
| PCC 4AMPyridine | 20 | 60 | 75 |
| Load pH 8.0 | | | |
| P CDI 4AMPyridine | 18.2 | 51 | 67 |
| Load pH 8.0 | | | |
| PCC APP 67/API 33 | 13.5 | 41 | 44 |
| Load pH 8.0 | | | |

*Assuming A_{280 nm} of 1.0 is equivalent to 1 mg/ml.
**Bicinchonic acid protein assay, BSA Standard.

The data in Table 2 demonstrate that the above resins have good capacity binding capacity for subtilisin.

### Example VIII

### Titration of Hydrophobic Positively Ionizable Resins

A sample of the resin (1 g; wet weight) to be titrated was washed with 0.1 M NaOH and then rinsed with water. The sample was then suspended in 9 ml of 500 mM NaCl solution (or as noted in Table 3) and titrated with 0.1 N HCl. Titration figures were corrected for dilution effects by subtracting titration values obtained for a underivatized Perloza control. The titration data is shown in Table 3.

TABLE 3
Titration Data

| | | pH Uncharged | |
|---|---|---|---|
| | | Form Starts to | pH 90% |
| Resin | Resin pKa | Titrate | Charged |
| PCC DEAPA | 9.3 | 10.5 | 8.4 |
| P CDI APMorpholine | 7.1 | 8.9 | 6.2 |
| PCC APImidazole | 6.25 | 8.0 | 4.8 |
| P CDI AMBenzimidazole | 4.8 | 6.75 | 4.0 |
| P CDI 2AMPyridine | 4.1 | 6.1 | 3.0 |
| P CDI 4AMPyridine | 4.7 | 7.2 | 3.7 |
| PCC 3AMPyridine | 4.2 | 7.0 | 3.3 |
| PCC API (10 mM NaCl) | 5.0 | 8.0 | 3.6 |
| P CDI APM (10 mM NaCl) | 5.8 | 8.05 | 4.8 |
| P CDI AMB (10 mM NaCl) | 3.6 | 6.45 | 3.0* |
| S ECH Methimazole | 5.6 | 7.6 | 4.5 |
| S ECH 4-Mercaptopyridine | 5.35 | 7.6 | 4.2 |
| S ECH Mercaptobenzimidazole | 4.2 | 6.5-7.0 | 3.1 |

*At low pH values, greater error is likely due to acid dilution.

### Example IX

### Titration of Hydrophobic Negatively Ionizable Resins

A sample of the resin (Ig) in 0.5 M NaCl was adjusted to pH 12 with 1 M NaOH and then titrated to pH 3 with 0.1 M HCl. Titration figures were corrected for dilution effects by subtracting titration values obtained for a underivatized Perloza control. The titration data are shown in Table 4.

TABLE 4
Titration Data

| | | pH Uncharged | |
|---|---|---|---|
| | | Form Starts to | pH 90% |
| Resin | Resin pKa | Titrate | Charged |
| P CDI DAH | 6.4 | 4 | 7.5 |
| Nitrohyroxyphenylacetic Acid | | | |
| P CDI Nitrotyrosine/ethanolamine | 7.2 | 5 | 9.0 |
| P CDI DAH Dichlorosalicylic Acid | 7.2 | 5 | 9.3 |
| P CDI Dibromotyramine | 7.7 | 5 | 9.3 |
| P CDI DAH | 9.8 | 6.5 | 10.8 |
| Chlorohydroxyphenylacetic Acid | | | |
| P CDI DAH Hydroxyphenylacetic | 10.7 | 7.5 | 11.2 |
| Acid | | | |
| P CDI Tyramine | 10.7 | 7.5 | 11.2 |
| PCC | 5.2 | 3.0 | 6.5 |
| PCC (10 mM NaCl) | 6.1 | 3.3 | 7.5 |

The data in Tables 3 and 4 show the pH range in which representative resins having an inducible charge are converted from a electrostatically uncharged state to an electrostatically charged state. This data illustrate that one skilled in the art may prepare and select various resins having different ionization profiles thereby permitting the use of a resin which is compatible with the target protein to be recovered.

### Example X

### Recovery of Subtilisin Using Batch Binding

A. Recovery Using API Substituted Perloza (MG1)

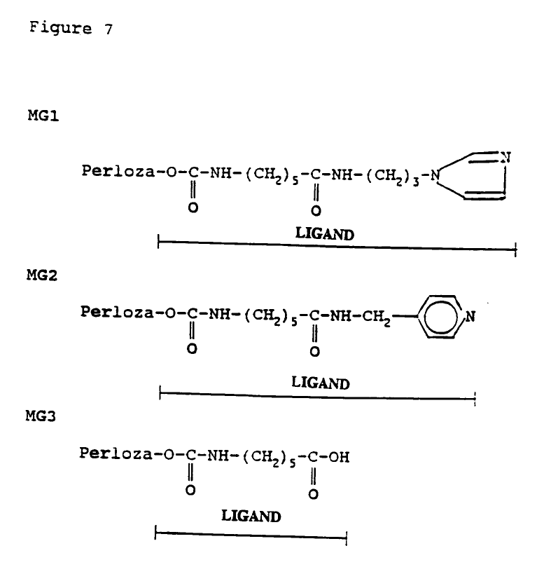

1-(3-Aminopropyl)imidazole-substituted Perloza (MG1) resin was prepared using an original matrix of Perloza MT 100 fine (available from Secheza, Prague, Czechoslovakia). Swollen volume of prepared resin is 6.8 ml per gram dry resin. The activation chemistry used was carbonyl diimidazole-aminocaproic acid activation. The activation substitution was 1.55 mM caproic acid groups/g. The ionizable functionality used was 1-(3-aminopropyl)imidazole, with substitution of 95-100% of caproic carboxyl groups. The structure of the resin is shown in FIG. 7.

The effect of salt and time on the binding of subtilisin broth to MG1 resin was examined. The high salt condition included the addition of 0.5 M NaCl to the Equilibration/Wash buffer. The low salt condition had no extra NaCl. The effect of salt addition to the elution buffer was also examined. The experimental conditions were as follows:

| | LOW SALT | HIGH SALT |
|---|---|---|
| Equilibration/Wash | 50 mM Glycine | 50 mM Glycine/0.5 M NaCl |
| Buffer: | pH 9.1 | pH 9.1 |
| | 0.8 mmho | 48.7 mmho |
| Elution Buffer: | 50 mM Acetate | 50 mM Acetate |
| | pH 5.2 | 0.5 M NaCl |
| | 4.3 mmho | pH 5.2/52.6 mmho |

| | |
|---|---|
| Regeneration: | Propylene Glycol/Ethanol/0.1 N HCl |
| Broth: | Broth filtered to 20 microns pH 9 |
| | 18 mmho |

Four grams of wet resin were equilibrated with high salt or low salt equilibration buffer up to a total volume of 10 mL. The settled volume of the resin was recorded before the experiment.

Fifty mL of fermentation broth containing a subtilisin varient, obtained as described in U.S. Ser. No. 137,240, filed Oct. 14, 1993, the entire disclosure of which is incorporated herein by reference, was adjusted to the proper pH. At time 0, the 10 mL of buffer and resin were added to the broth. An additional 1 mL of water was added to rinse the test tube. The mixture was stirred in a beaker of known weight under refrigeration for 5 minutes. At that time, a portion of the supernatant was filtered through a Buchner funnel containing Whatman 47 filter paper. A 0.1 mL sample was collected. Then the supernatant and resin were once again contacted for an additional 30 minutes.

At that time all supernatant was separated from the resin by filtration. The resin was rinsed with Equilibration/Wash buffer, then placed back in the beaker and contacted with 100 mL of Wash buffer to remove any non-selectively binding components. The solution was again filtered. The resin was then contacted with 100 mL of elution buffer for at least one hour.

After elution, the resin was contacted with propylene glycol for at least 10 minutes. This was followed by a brief ethanol rinse. Finally, the resin was contacted with 0.1 M HCl regeneration buffer for at least 15 minutes.

Samples of the supernatants and elution fractions were analyzed for activity. More subtilisin was bound to the resin after 35 minutes as compared to that bound after 5 minutes. The results after 35 minutes are shown in Table 5.

TABLE 5
MG1 Batch Binding of Subtilisin

| | Low Salt | High Salt |
|---|---|---|
| Volume of Resin (ml) | 6.0 | 6.0 |
| Total Active Enzyme | | |
| Contacted with Resin (mg) | 343.99 | 328.50 |
| Total Unbound Enzyme (mg) | 192.06 | 175.74 |
| Total Bound Enzyme (mg) | 151.93 | 152.76 |
| Total Eluted Enzyme (mg) | 19.35 | 62.63 |
| Total Enzyme Not Recovered (mg) | 132.58 | 90.13 |
| Resin Capacity (mg/ml) | 25.32 | 25.46 |

The data in Table 5 demonstrate that MG1 resin bound approximately the same amount of subtilisin at high and low ionic strength, with a capacity of about 25 mg/ml under either condition. Elution efficiency was better using high salt elution buffer.

B. Recovery Using 4AMP Substituted Perloza (MG2)

4-(Aminomethyl)pyridine-substituted Perloza (MG2) resin was prepared using an original matrix of Perloza MT 100 fine. Swollen volume of prepared resin was 6.8 ml per gram dry resin. The activation chemistry used was carbonyl diimidazole-aminocaproic acid activation. The activation substitution was 1.55 mM caproic acid groups/g. The ionizable functionality used was 4-(aminomethyl)pyridine, with substitution on 95-100% of caproic carboxyl groups. The structure of the resin is shown in FIG. 7.

The effect of salt concentration on the binding of subtilisin to MG2 resin was examined. The high salt condition included the addition of 0.5 M NaCl to the Equilibration/Wash buffer. The low salt condition had no extra NaCl. The same elution buffer was used for both. The experimental conditions were as follows:

| | LOW SALT | HIGH SALT |
|---|---|---|
| Equilibration/Wash Buffer: | 50 mM Tris | 50 mM Tris/0.5 M NaCl |
| | pH 7.7 | pH 7.7 |
| | 3.3 mmho | 51.2 mmho |
| Elution Buffer 1 & 2: | 100 mM Acetate | 100 mM Acetate |
| | pH 5.2 | pH 5.2 |
| | 8.2 mmho | 8.2 mmho |

| | |
|---|---|
| Regeneration: | Propylene Glycol/Ethanol/0.1 N HCl |
| Broth: | Broth filtered to 20 microns pH 7.7 |
| | 16.8 mmho |

Procedures followed were as in EXAMPLE X-A above, except that the sample was contacted with the first elution buffer for 90 minutes and with the second elution buffer for 30 minutes.

Samples of the supernatants and elution fractions were analyzed for total protein content and activity. Results after 35 minutes are shown in Table 6.

TABLE 6
MG2 Batch Binding of Subtilisin

| | Low Salt | | | | | |
|---|---|---|---|---|---|---|
| | Tot. | | | High Salt | | |
| | Pro. | Enzyme | Other | Tot. Pro. | Enzyme | Other |
| Protein | 1552.27 | 431.86 | 1120.40 | 1709.95 | 467.38 | 1242.57 |
| Contacted | | | | | | |
| with Resin | | | | | | |
| (mg) | | | | | | |
| Unbound | 1092.24 | 219.99 | 872.25 | 990.03 | 213.17 | 776.86 |
| Protein (mg) | | | | | | |
| Total Bound | 460.02 | 211.87 | 248.16 | 719.92 | 254.21 | 465.71 |
| (mg) | | | | | | |
| Elution E1 | 343.86 | 153.79 | 190.08 | 403.54 | 161.29 | 242.25 |
| Elution E2 | 26.98 | 11.57 | 15.41 | 18.75 | 12.14 | 6.61 |
| Total | 104.58 | 46.51 | 58.07 | 333.49 | 80.78 | 233.97 |
| Protein Not | | | | | | |
| Recovered | | | | | | |
| (mg) | | | | | | |
| Resin | 73.02 | 33.63 | 39.39 | 128.56 | 45.39 | 83.17 |
| Capacity | | | | | | |
| (mg/ml) | | | | | | |
| Resin Vol. | | 6.3 | | | 5.6 | |
| (ml) | | | | | | |

The data in Table 6 illustrate that MG2 has an active enzyme capacity of about 33 mg/ml at low salt and about 42 mg/ml at high salt. The total protein 30 capacity is significantly higher at 73 and 129 mg/ml respectively, Although hydrophobic binding is improved at higher salt concentration, approximately 30% of the total protein and approximately 50% of the target protein was bound at low salt concentrations. Elution was more efficient than MG1, and the second elution did not remove much additional subtilisin.

### Example XI

### Recovery of Subtilisin Using Aminomethyl Pyridine Substituted Perloza (MG2)

A. Recovery in a 50 in L Radial Flow Column

Perloza MT100 fine regenerated cellulose matrix (Secheza, Prague. Czechoslovakia) was activated with CDI according to Example VI. It was substituted with 4-aminomethlylpyridine as the ionizable functionality using an aminocaproic acid spacer arm. Swollen volume of the prepared resin was 6.8 mL/g dry resin. Ionizable functionality substitution was 95-100% of available caproic carboxyl groups. The structure of the resin is shown in FIG. 7.

A 50 mL radial flow chromatography column (Sepragen Corp., San Leandro, Calif.) was packed with the above-prepared resin and used to recover PURAFECT™ subtilisin, commercially available from Genencor International. Inc. South San Francisco. Calif. as described below:

Broth Treatment:

The whole broth was centrifuged for 45 min. in a chilled Sorvall centrifuge at 4,500 rpm. The conductivity of the centrate was not adjusted, and no dilutions were made. It was filter to remove particles greater than 10 microns. The pH of the broth was not adjusted (it was already 7.6). The conductivity of the broth was 16 mmho. The broth contained 8.9 mg/ml of active protein, as well as several contaminating proteins.

| Buffers: | |
|---|---|
| Equilibration Buffer: | 50 mM TRIS ™; pH 7.8; + NaCl to 17 mmho |
| Wash Buffer: | 50 mM TRIS ™; pH 7.8; + NaCl to 17 mmho |
| Elution Buffer: | 25 mM Acetate, 40% Propylene Glycol, 8% |
| | Sodium Formate, pH 5.2, 30 mmho |
| Regeneration Buffer: | 0.1 N HCl, pH 1.9, 30 mmho |

Centrifuged broth (251 ml) was loaded onto the column at pH 7.8 and a conductivity of 16-18 mmho (equivalent to an NaCl concentration of 0.15-0.18 M). The flow rate was 10 ml/min. Subtilisin bound to the uncharged resin, forming a resin/protein complex. The column was washed for 25 column volumes at 15 ml/min and eluted over 15 column volumes at 5 ml/min. There was minimal active enzyme detected in the flow through fractions, 93% subtilisin formed a complex with resin. 42% of the bound enzyme was lost during the wash. The pressure throughout the run was below 10 psi.

This complex was broken and the enzyme eluted by decreasing the pH and increasing the conductivity using the above elution buffer. 91% of the bound enzyme was recovered. The maximum fraction concentration was 12.8 mg/ml, and 90% of the recovered enzyme eluted within 4 CVs. Because of the wash losses, the overall recovery was 48.6%. The ratio of active enzyme to total protein showed that the most concentrated elution fractions were 15-25% more pure than the feed.

B. Recovery in a 3.5 in L Axial Flow Column

A 3.5 ml axial flow chromatography column (Pharmacia) was packed with the same resin and used to recover subtilisin from similar broth, using different broth treatment and somewhat different buffers.

Broth Treatment:

The whole broth was centrifuged for 45 min. in a chilled Sorvall centrifuge at 4.500 rpm. The conductivity of the centrate was not adjusted, and no dilutions were made. It was filtered to remove particles greater than 10 microns. The pH of the broth was not adjusted (it was already 7.6). The conductivity of the broth was 14 mmho. The broth contained 9.8 mg/ml of active protein, as well as several contaminating proteins.

| Buffers: | |
|---|---|
| Equilibration Buffer: | 50 mM TRIS ™ + NaCl to 14 mmho; pH 7.6 |
| Wash Buffer: | 50 mM TRIS ™ + NaCl to 14 mmho; pH 7.6 |
| Elution Buffer: | 25 mM Acetate, 40% propylene glycol, 0.8% |
| | Sodium Formate, pH 5.2, 5 mmho |
| Regeneration Buffer: | 0.1 N HCl |

Broth centrate (14.4 ml) was loaded onto the column at pH 7.6 and a conductivity of 14 mmho (equivalent to a total NaCl concentration of 0.13 M). The flow rate was 0.85 CV/min. Subtilisin bound to the uncharged resin, forming a resin/protein complex. The column was washed for 155 column volumes and eluted over 93 column volumes. There was no active enzyme detected in the flow through fractions. Therefore, all subtilisin formed a complex with the resin. There was a 32% loss of bound enzyme in the very long wash. The pressure throughout the run was below 15 psi.

This complex was broken and the enzyme eluted by decreasing the pH using the above elution buffer (equivalent to an NaCl concentration of 0.03 M). 86% of the bound enzyme was recovered. The total subtilisin recovery was 58% when wash losses were included.

It will be apparent to one of ordinary skill in the art that various changes and modifications of an obvious nature may be made without departing from the spirit of the invention, and all such modifications are considered to fall within the scope of the invention, as set forth in the following claims.

1. A resin-protein/peptide complex which comprises a resin and a target protein or peptide bound thereto wherein said resin comprises
a) a solid support matrix; and
b) selected ionizable ligand covalently attached to the matrix
wherein the ionizable ligand is selected such that the resin is electrostatically uncharged at a high and a low ionic strength at the pH where the target protein or peptide is bound to the resin wherein the protein or peptide binds to the resin at a pH of 5 to 9 and is electrostatically charged at the pH where the target protein or peptide is desorbed from the resin wherein desorption occurs by a change in the pH from the binding pH and further wherein about 50 percent or more of the target protein or peptide in an aqueous medium binds to the resin when the aqueous medium has either a high or a low ionic strength.

2. The resin-protein complex of claim 1 wherein the ionizable ligand is electrostatically uncharged at the pH where the target protein or peptide is bound to the resin and is positively charged at the pH where the target protein or peptide is desorbed from the resin.

3. The resin-protein/peptide complex of claim 1 wherein the ionizable ligand is electrostatically uncharged at the pH where the target protein or peptide is bound to the resin and is negatively charged at the pH where the target protein or peptide is desorbed from the resin.

4. The resin-protein/peptide complex of claim 1 wherein the ionizable ligand comprises an ionizable functional group directly attached to the solid support matrix.

5. The resin-protein/peptide complex of claim 1 wherein the ionizable ligand comprises a spacer arm and at least one ionizable functionality wherein the ionizable functionality is attached to the solid support matrix via the spacer arm.

6 (Canceled)

7. The resin-protein/peptide complex of claim 1 wherein the resin further comprises non-ionizable ligands.

8. The resin-protein/peptide complex of claim 7 wherein the percentage of non-ionizable ligands attached to the solid support matrix based on the total of ionizable and non-ionizable ligands ranges from greater than 0% to about 80%.

9. The resin-protein/peptide complex of claim 8 wherein the percentage of non-ionizable ligands attached to the solid support matrix based on the total of ionizable and non-ionizable ligands ranges from greater than 0% to about 40%.

10. The resin-protein/peptide complex of claim 1 wherein the solid support matrix is cross-linked.

11. The resin-protein/peptide complex of claim 1 wherein the resin contains from about 0.05 mmol to about 0.5 mmol ionizable ligand per ml of the solid support matrix prior to covalent attachment of any non-ionizable ligand.

12. The resin-protein/peptide complex of claim 1 wherein the solid support matrix is non-ionizable.

13. The resin-protein/peptide complex of claim 1 wherein the solid support matrix contains ionizable functionality which functionality is electrostatically uncharged at the pH where the target protein or peptide is bound to the resin and is electrostatically charged at the pH where the target protein or peptide is desorbed from the resin.

14. The resin-protein/peptide complex of claim 1 wherein the electrostatic charge induced on the resin of the resin-protein/peptide complex is of the same polarity as the net electrostatic charge on the target protein or peptide at the pH of desorption.

15. The resin-protein/peptide complex of claim 1 wherein the electrostatic charge induced on the resin of the resin-protein/peptide complex is of the opposite polarity from the net electrostatic charge on the target protein or peptide at the pH of desorption.

16. A resin-protein/peptide complex which comprises a resin and a target protein or peptide bound thereto wherein said resin comprises
a) a solid support matrix having a selected ionizable functionality incorporated into the backbone thereof wherein the ionizable functionality is selected such that the resin is electrostatically uncharged at a high and a low ionic strength at the pH where the target protein or peptide is bound to the resin wherein the protein or peptide binds to the resin at a pH of 5 to 9 and is electrostatically charged at the pH where the target protein or peptide is desorbed from the resin wherein desorption occurs by a change in the pH from the binding pH; and
b) optionally a non-ionizable ligand covalently attached thereto,
wherein about 50 percent of more of the target protein or peptide in an aqueous medium binds to the resin when the aqueous medium has either a high or a low ionic strength.

17. The resin-protein/peptide complex of claim 16 wherein the ionizable functionality is electrostatically uncharged at the pH where the target protein or peptide is bound to the resin and is positively charged at the pH where the target protein or peptide is desorbed from the resin.

18. The resin-protein/peptide complex of claim 16 wherein the ionizable functionality is electrostatically uncharged at the pH where the target protein or peptide is bound to the resin and is negatively charged at the pH where the target protein or peptide is desorbed from the resin.

19. The resin-protein/peptide complex of claim 16 wherein the ionizable functionality comprises amino groups covalently attached in the backbone of the solid support matrix.

20. The resin-protein/peptide complex of claim 16 wherein the solid support matrix is cross-linked.

21. The resin-protein/peptide complex of claim 16 wherein the resin contains from about 0.05 mmol non-ionizable ligand per ml of the solid support matrix.

22. The resin-protein/peptide complex of claim 16 wherein the electrostatic charge induced on the resin of the resin-protein/peptide complex is of the same polarity as the net electrostatic charge on the target protein or peptide at the pH of desorption.

23. The resin-protein/peptide complex of claim 16 wherein the electrostatic charge induced on the resin of the resin-protein/peptide complex is of the opposite polarity from the net electrostatic charge on the target protein or peptide at the pH of desorption.

24-54. (Canceled)

55. A resin-protein/peptide complex which comprises a resin and a target protein or peptide bound thereto wherein said resin comprises
a) a solid support matrix; and
b) selected ionizable ligand covalently attached to the matrix,
wherein the ionizable ligand is selected such that the resin is electrostatically uncharged at a high and a low ionic strength at the pH where the target protein or peptide is bound to the resin wherein the protein or peptide binds to the resin at a pH of 5 to 9 and is electrostatically charged at the pH where the target protein or peptide is desorbed from the resin wherein desorption occurs by a change in the pH from the binding pH, and wherein said ionizable ligand is selected from group consisting of amine groups, phenolic groups, histidyl groups, hydroxyl groups, pyridyl groups, anilino groups, morpholinyl groups, thiol groups, and imidazolyl groups, and further wherein about 50 percent or more of the target protein or peptide in an aqueous medium binds to the resin when the aqueous medium has either a high or low ionic strength.

56. A resin-protein/peptide complex which comprises a resin and a target protein or peptide bound thereto wherein said resin comprises
a) a solid support matrix having a selected ionizable functionality incorporated into the backbone thereof wherein the ionizable functionality is selected such that the resin is electrostatically uncharged at a high and a low ionic strength at the pH where the target protein or peptide is bound to the resin wherein the protein or peptide binds to the resin at a pH of 5 to 9 and is electrostatically charged at the pH where the target protein or peptide is desorbed from the resin, wherein desorption occurs by a change in the pH from the binding pH, and further wherein said ionizable ligand is selected from group consisting of amine groups, phenolic groups, histidyl groups, hydroxyl groups, pyridyl groups, anilino groups, morpholinyl groups, thiol groups, and imidazolyl groups; and
b) optionally a non-ionizable ligand covalently attached thereto,
wherein about 50 percent or more of the target protein or peptide in an aqueous medium binds to the resin when the aqueous medium has either a high or low ionic strength.
